(19) United States
(12) Patent Application Publication
(10) Pub. No.: US 2021/0270814 A1
(43) Pub. Date: Sep. 2, 2021

(54) Methods for Identifying Two or More Populations Having Different Responses to Chemical or Biological Agents

(71) Applicant: Coyne Scientific, LLC, Atlanta, GA (US)

(72) Inventors: Kevin P. Coyne, Atlanta, GA (US); Shawn T. Coyne, Smyrna, GA (US)

(21) Appl. No.: 16/075,178

(22) PCT Filed: Feb. 3, 2017
(86) PCT No.: PCT/US17/16441

§ 371 Date: Aug. 3, 2018

Related U.S. Application Data
(60) Provisional application No. 62/290,548, filed on Feb. 3, 2016.

(51) Int. Cl. G01N 33/50

(52) U.S. Cl. CPC G01N 33/5073; G01N 33/5014

(57) ABSTRACT

Cell lines, such as iPSC cell lines, or other tissue constructs created from tissue samples collected from large numbers of donors, are used in assessing the biological reactions of individuals to biological or chemical agents. The donor tissue samples are grouped according to a phenotypic or genotypic trait that is exogenous to the results of the assays conducted. Cells or tissues derived from the samples of each of the individuals are combined with one or more chemical or biological agents and tested in identical assays to determine the impact of the chemical or biological agents. The results of each separately defined phenotypic or genotypic subgroup are aggregated to form a distribution of impacts for that particular group. The distributions associated with the various groups are compared to determine whether there is a statistically meaningful difference in the responses of the groups.

### CROSS REFERENCE TO RELATED APPLICATIONS

This application claims the benefit of U.S. Provisional Application No. 62/290,548, filed on Feb. 3, 2016, which is incorporated by reference herein in its entirety.

### BACKGROUND

It is well established in the scientific literature that certain phenotypically or genotypically defined populations exhibit important differences in biological activity. For example, researchers have identified race and gender as two phenotypic populations whose responses to pharmaceuticals are importantly different (Fakunle, E S. Loring, J F. Trends in Molecular Medicine. 18 (12): 709-716 (2012); O'Shea, S H. et al. Toxicological Sciences. 119 (2): 398-407 (2011); Abdo, N. et al. Environmental Health Perspectives. 123 (5): 458-466 (2015)).

Other populations have also exhibited differences in biological reactions that are seemingly unrelated to a phenotypic trait that distinguishes them. In one recent example, a study found that persons with light colored eyes exhibit higher incidences of alcoholism than persons with darker colored eyes (Sulovari, A et al., American Journal of Medical Genetics: Neuropsychiatric Genetics (Part B). 9999B:1-7 (2015)), believed to be due to the proximity of genes controlling eye color to other genes affecting predisposition to alcoholism.

However, discovering newly defined populations that exhibit different biological reactions than their counterparts has been difficult and time consuming. For example, in the field of drug toxicity testing, researchers have historically relied on data from clinical trials. However, such data is limiting, for several reasons. First, the data may consist of only simple outcomes (e.g., binary counts of patients reporting an effect versus those reporting no effect). Thus, the severity of an adverse reaction is often not quantitatively measured in the clinic. This is problematic because different distributions of severity of reactions between subpopulations can be an important indicator of biological differences that would not be revealed by a comparison of incidence only.

Second, clinical data is almost always “contaminated' by confounding factors, such as differences among the patients in age, prior health, differing consumption of medicines that are not the subject of the trial, etc.

Third, by definition, each trial participant can experience only one dose regimen of the compound under study. Thus, any attempt to compare differences among populations across dose regimens is limited to clinical trials involving very large sample sizes, and limited by the actual dose regimens tested.

Therefore, the field of biology would benefit from the ability to identify sample populations based on phenotypic or genetic differences and contrast the degrees of reaction (in an experimentally robust way) by the members of those sub-populations such that the results for the various populations can be compared and contrasted based on the distributions of the degree of their reactions to a particular stimulus rather than on a binary case-versus-control basis.

Recent scientific advances in the development of stem cells have theoretically opened the possibility of using in vitro testing to conduct such studies. For example, stem cells and their derivatives enable large scale production of sufficient quantities of cells from the same individual to conduct multiple experiments.

To-date, however, such cells have not been used for this purpose. Comparing results across individuals in sufficient numbers to support conclusions at the level of sub-populations depends on the development of large scale cohorts of cell lines that are considered to be sufficiently biologically comparable to each other (e.g., developed under similar isolation, culturing and differentiation conditions) to support cross-donor comparisons of results. The development of such platforms has been described in the following three related patent applications (PCT/US14/45499, entitled “Methods for Predicting Responses to Chemical or Biologic Substances”; PCT/US14/53819, entitled “Methods for Genetically Diversified Stimulus-Response Based Gene Association Studies”; and PCT/US15/55637, entitled “Methods For Conducting Stimulus-Response Studies With Induced Pluripotent Stem Cells Derived from Perinatal Cells or Tissues”), which are each incorporated by reference herein in their entireties.

The methods provided herein build on the capability to create a large sample of cohort of cell lines by specifying the design and subsequent processes to examine whether the reactions to chemical or biological agents do indeed vary significantly across such populations when both incidence and severity of reaction are considered.

### SUMMARY

Embodiments of the present disclosure comprise methods to identify populations or sub-populations having significantly different responses to stimuli, for example, exposure to a biological or chemical agent.

Methods for identifying two or more populations of subjects whose reactions to chemical or biological agents vary significantly from each other using in vitro testing are provided herein. The methods include, in any appropriate order, the steps of: (1) identifying one or more phenotypic or genotypic traits of subjects to test for at least one biological response to a chemical or biological agent that differs from subjects who possess one or more alternative phenotypic or genotypic traits; (2) identifying sufficiently large and representative samples of a population who possess the phenotypic or genetic traits to be contrasted, but who otherwise are deemed to be similar enough that any differences in biological reactions are deemed to be due to the differences in the traits of interest only; (3) obtaining, culturing, and/or manipulating cells or tissues from these samples under a common, rigorous protocol that enables comparability of findings of the tests to be administered across the subjects; (4) testing in vitro the cells or tissues from each of those samples, or cells or tissues derived from those samples, such as induced pluripotent stem cells or their derivatives, under challenge by a chemical or biological agent of interest, and quantifying results; and (5) comparing the distributions of the quantitative endpoint results at the individual level of the in vitro assays of any one of the samples defined in step (2) above to the distribution of the quantitative endpoint results of the same in vitro assays conducted on any of the other samples, and employing statistical techniques to determine whether the samples are distinct in their reactions to a statistically meaningful degree.

More specifically, methods described herein include methods for identifying two or more populations having a statistically significant difference in a response to exposure to a chemical or biological agent, comprising a) exposing the chemical or biological agent to samples collected from subjects of a first sub-population and samples collected from subjects of a second sub-population for a time sufficient for a response to be observed, wherein one or more phenotypic or genotypic traits of the subjects of the first sub-population are different from the phenotypic and genotypic traits of the second sub-population and are suspected of being related to one or more responses to exposure to the agent; b) assaying the samples of a) exposed to the agent for a response; c) quantifying an endpoint of the response for each sample; d) providing a distribution of endpoints quantified in step c) for each of the first and second sub-populations samples; e) comparing the distribution of endpoints of the first sub-population samples to the distribution of endpoints of the second sub-population samples; and f) determining whether the response of the first sub-population samples to exposure to the agent is statistically different from the response of the second sub-population samples to exposure to the agent.

Also disclosed are methods for determining whether a first subpopulation exposed to radiation with have a statistically significant difference in a response to exposure to a chemical or biological agent compared to a second subpopulation, comprising: a) exposing the chemical or biological agent to samples collected from subjects of a first sub-population exposed to toxic levels of radiation and samples collected from subjects of a second sub-population not exposed to toxic levels of radiation for a time sufficient for a response to be observed; b) assaying the samples of a) exposed to the agent for a response; c) quantifying an endpoint of the response for each sample; d) providing a distribution of endpoints quantified in step c) for each of the first and second sub-populations samples; e) comparing the distribution of endpoints of the first sub-population samples to the distribution of endpoints of the second sub-population samples; and f) determining whether the response of the first sub-population samples to exposure to the agent is statistically different from the response of the second sub-population samples to exposure to the agent.

Also disclosed herein are methods for determining whether race or ethnicity of a first subpopulation with result in a statistically significant difference in a response to exposure to a chemical or biological agent compared to a second subpopulation, comprising: a) exposing the chemical or biological agent to samples collected from subjects of a first sub-population and samples collected from subjects of a second sub-population for a time sufficient for a response to be observed, wherein the subjects of the first sub-population differ in race or ethnicity from the subjects of the second sub-population; b) assaying the samples of a) exposed to the agent for a response; c) quantifying an endpoint of the response for each sample; d) providing a distribution of endpoints quantified in step c) for each of the first and second sub-populations samples; e) comparing the distribution of endpoints of the first sub-population samples to the distribution of endpoints of the second sub-population samples; and f) determining whether the response of the first sub-population samples to exposure to the agent is statistically different from the response of the second sub-population samples to exposure to the agent.

Also disclosed herein are methods for identifying whether two or more populations have a statistically significant difference from each other in a response to a chemical or biological agent, comprising a) providing sample cells that can be reprogrammed into iPSCs from a sample of subjects of a first sub-population who possess one or more phenotypic or genotypic traits suspected of causing or being co-incidentally related to one or more biological reactions to the chemical or biological agent; b) providing sample cells that can be reprogrammed into iPSCs from a sample of subjects of a second sub-population having one or more phenotypic or genotypic traits that are different from those of the first sub-population, and wherein these traits are suspected of causing or being co-incidentally related to one or more biological reactions to the chemical or biological agent; c) optionally providing sample cells that can be reprogrammed into iPSCs from a sample of subjects of additional sub-populations that meet the same criteria as in step b); d) reprogramming the cells from each subject of the sub-populations in a) through c) into iPSCs, and optionally, further differentiating the iPSCs into functional cells; e) for each sample from each subject, combining the chemical or biological agent in vitro with the cells developed in step d); f) assaying for an effect of result of the reaction for each member of each sub-population; g) quantifying an endpoint of the effect of each sample; h) comparing the distribution of endpoint scores of the effect on subjects from the first sub-population to the distribution of endpoints of the effect on subjects from the second sub-population, and, optionally, any additional sub-populations; and i) employing statistical techniques to determine whether the effect of the chemical or biological agent on subject samples from the first sub-population are statistically different from the effect on subject samples from the second and/or additional sub-populations.

### DETAILED DESCRIPTION

Titles or subtitles may be used in the specification for the convenience of a reader, which are not intended to influence the scope of the present invention.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. It will be further understood that the endpoints of each of the ranges are significant both in relation to the other endpoint, and independently of the other endpoint. Thus, the endpoint of one range is combinable with the endpoint of another range. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 samples refers to groups having 1, 2, or 3 samples. Similarly, a group having 1-5 samples refers to groups having 1, 2, 3, 4, or 5 samples, and so forth.

### DEFINITIONS

The following terms, unless otherwise indicated, shall be understood to have the following meanings:

As used herein, the terms “a”, “an”, and “the” can refer to one or more unless specifically noted otherwise.

The use of the term “or” is used to mean “and/or” unless explicitly indicated to refer to alternatives only or the alternatives are mutually exclusive, although the disclosure supports a definition that refers to only alternatives and “and/or.” As used herein “another” can mean at least a second or more.

Throughout this application, the term “about” is used to indicate that a value includes the inherent variation of error for the device, the method being employed to determine the value, or the variation that exists among samples. It is to be understood, although not always explicitly stated, that all numerical designations may be preceded by the term “about.”

The term “comprising” or “comprises” is intended to mean that the methods include the recited elements, but not excluding others. “Consisting essentially of” when used to define methods, shall mean excluding other elements of any essential significance to the combination. For example, a method consisting essentially of the elements as defined herein would not exclude other elements that do not materially affect the basic and novel characteristic(s) of the claimed method. “Consisting of” shall mean excluding more than trace amount of other ingredients and substantial method steps. Embodiments defined by each of these transition terms are within the scope of this invention.

The term “sub-population” refers to any group of subjects which comprise from greater than zero percent to less than 100 percent of a population of subjects. There is no requirement that a population be identified a priori and then sub-divided. In fact, each “sub-population” may be identified entirely independently of all other sub-populations, and a decision to practice the present invention may be made subsequent to the identification of all, or some, of the sub-populations. Further, any sub-population may be compared to another sub-population. For instance, the terms “first sub-population” and “second sub-population” refer to any sub-populations whose responses may be compared. There may be any number of such sub-populations, and the definitions of such sub-populations are not restricted to being non-overlapping. Thus a reference to comparing results of a first sub-population to a second sub-population may actually involve comparisons of any number of sub-populations, and may involve two or more sub-populations that were identified in any order. For example, a comparison of the results for the first sub-population to the results for the second sub-population may actually involve a multi-way comparison among the fourth, seventeenth, twenty-third and thirty-fifth sub-populations defined.

The term “tissue” (or “tissues”) as used herein includes biological cells, tissues and fluids containing cells, preferably cells, tissues and fluids containing cells that are multi-potent, or that can be used to create induced pluripotent stem cells (iPSCs), or functional cells derived from iPSCs. The cells can be obtained from large numbers of subjects as long as the cells have been completely reprogrammed to a pluripotent state via a non-integrating method. Tissues of any kind can be collected, such as, but not limited to, amniotic fluid; cord blood or tissue; placenta; peripheral blood cells; cells obtained from skin (including dermal fibroblasts) or hair; and cells obtained through a swab of the cheek or any alimentary passage of the donor.

The term “donor” (or “donors”) and “subjects” (or “subjects”) as used herein includes any human or other animal of any age and are used interchangeably. The term includes any vertebrate, more specifically a mammal (e.g. a human, horse, cat, dog, cow, pig, sheep, goat, mouse, rabbit, rat, and guinea pig), birds, reptiles, amphibians, fish, and any other animal. The term does not denote a particular age or sex. Thus, adult and newborn subjects, whether male or female, are intended to be covered.

“Optional” or “optionally” means that the subsequently described event, circumstance, or material may or may not occur or be present, and that the description includes instances where the event, circumstance, or material occurs or is present and instances where it does not occur or is not present.

### Methods to Identify Populations Having Different Responses to Agents

Methods described herein include methods for identifying two or more populations having a statistically significant difference in a response to exposure to a chemical or biological agent, comprising a) exposing the chemical or biological agent to samples collected from subjects of a first sub-population and samples collected from subjects of a second sub-population for a time sufficient for a response to be observed, wherein one or more phenotypic or genotypic traits of the subjects of the first sub-population are different from the phenotypic and genotypic traits of the second sub-population and are suspected of being related to one or more responses to exposure to the agent; b) assaying the samples of a) exposed to the agent for a response; c) quantifying an endpoint of the response for each sample; d) providing a distribution of endpoints quantified in step c) for each of the first and second sub-populations samples; e) comparing the distribution of endpoints of the first sub-population samples to the distribution of endpoints of the second sub-population samples; and f) determining whether the response of the first sub-population samples to exposure to the agent is statistically different from the response of the second sub-population samples to exposure to the agent.

In one embodiment, the method is directed to the creation of a single sample population of donors, on which tissues from those donors are subjected to in vitro tests. The sample is later divided into sub-populations. Division into sub-populations can be based, for example, on contrasting phenotypic or genotypic traits that the researcher hypothesizes have differing responses to an agent of interest. By sub-dividing the pre-existing collections of endpoint results at the individual level into various sets of sub-populations (defined by contrasting phenotypic traits or genotypic traits) and comparing the distributions of results within each sub-population to those of the other sub-populations, the researcher can analytically test many hypotheses rapidly.

In a second embodiment, the individuals in the sample population are divided into sub-populations (based on phenotypic and/or genotypic distinctions) prior to the administration of the in vitro tests.

In a third embodiment, the researcher identifies a particular phenotypic or genotypic distinction being associated with (and preferably causing) differing reactions to an agent of interest first, and only then assembles separate samples from within a population that correspond to the phenotypic or genotypic distinctions.

As understood by one skilled in the art, isolation of variables is central to investigating the effects on, or effects emanating from, those tested variables. However, in some situations, complete isolation of a single variable is not possible or even plausible. For example, studies of whole human beings or interactions between human beings involve countless variables, all of which cannot be completely controlled even under the most stringent conditions. As such, population studies of animals such as humans and other mammals should be designed to isolate the tested variable and control for other variables to a degree acceptable to one skilled in the art for making scientific conclusions from the data derived therefrom. Thus, in some embodiments, the first and second sub-populations are sufficiently similar such that a significant difference in the response to exposure to the agent is primarily due to the one or more phenotypic or genotypic traits of the subjects of the first sub-population that are different from the phenotypic and genotypic traits of the second sub-population and are suspected of being related to one or more responses to exposure to the agent. For example, a first and second population may be similar in one or more characteristics including age, gender, race, ethnicity, socio-economic status, genotype or chromosomal markers, physical characteristics such as size, height, weight, hair/eye color, etc., and a number of other characteristics pertinent to the study, but be significantly different in the amount of toxic radiation the populations are exposed to.

The degree of difference between the one or more phenotypic or genotypic traits of the subjects of the first and second sub-population (or any additional sub-population being compared in the method) depends on the nature of the comparison to be made. In all instances, the degree of difference is limited only in that the difference must be observable, capable of being modeled, and/or measurable. Examples of differences in a phenotypic trait include, but are not limited to, differences at the molecular level (e.g. protein isotypes), cellular level (e.g. cellular metabolism or secretion), tissue level (e.g. concentration of melanin per unit of skin), organ level (e.g. liver bile production), and/or organism level (e.g. ethnicity). Differences in a genotypic trait include differences in deoxyribonucleic acid (DNA), which includes differences in the genetic sequence of a gene, regulatory region, non-coding region, etc. Gene variants are typically referred to as alleles, which can produce phenotypic variants.

Phenotypic or genotypic traits are suspected of being related to one or more responses to exposure to the agent if there is a hypothetical connection between the trait and the response explainable on any scientific foundation. A mere hunch that a trait is related to a response is sufficient if the hunch can be at least minimally supported in any way by rational, scientific understanding, even if extensive countervailing evidence is present.

Various samples can be collected from subjects and are not particularly limited. Samples can include, but are not limited to, cells (e.g. skin cells or oral swabs), tissues (e.g., biopsied tissue such as tumors), blood (e.g. cord blood), hair or nails, sputum, mucosal and other bodily secretions, etc. Samples may be obtained by technicians (e.g. phlebotomist) by any method known in the art, and can be collected via, for instance, donation banks, hospitals/clinics, or post-mortem. Samples are collected and stored under conditions to maintain the integrity of the sample and avoid contamination (e.g. in sterile cryo-tubes).

Generally, certain attributes of population studies depends in part on the number of objects (n) used in the study. Generally speaking, as the number (n) increases, so does the accuracy, statistical significance, and repeatability of the study. In some embodiments, the number of samples to be collected and analyzed in the herein described methods is preferably more than about 10, 20, 25, 100 or 300. The maximum total number of samples included in any disclosed method is not limited. The number of samples selected for use in the disclosed methods may be less than the total number of samples collected from subjects. The total number of samples included in any disclosed method depends in part on factors such as sample availability and integrity, and the selection criteria for populations and subpopulations.

In some embodiments, samples can include cells which can be reprogrammed in vitro to induced pluripotent stem cells (iPSCs). In some embodiments, a portion of the donated sample from a subject is reprogrammed to iPSCs, and the iPSCs are subjected to the methods disclosed herein. Where desirable, reprogrammed iPSCs may be differentiated into functional cells. In some embodiments a portion of the functional cells derived from iPSCs are subjected to the methods disclosed herein.

The agent can be any biological or chemical agent which can be exposed to a biological sample collected from a subject. Numerous agents may be of interest and hence, are not particularly limited. Non-limiting examples of agents include biological agents such as antibodies, proteins, lipids and glycolipids, steroids, hormones, neurotransmitters, viruses, viral vectors, bacteria, liposomes, biological extracts such as plant extracts, and chemical agents such as small molecules, carbon-based molecules, synthetic and derivative molecules, drugs such as therapeutic drugs, and a wide range of other agents. For example, certain therapeutic and/or pharmaceutical compounds may be of interest to a researcher for a particular situation, but a different set of therapeutic and/or pharmaceutical compounds may be of interest in a different situation. As an example, in situations in which it is desirable to investigate which agents are effective to treat radiation exposure, the agent can be an FDA-approved or experimental drug administrable to treat radiation exposure. In situations in which it is desirable to investigate which agents are effective to treat a condition but avoid cardiotoxicity, the agent can be an FDA-approved or experimental drug administrable to treat that condition that is evaluated for cardiotoxic effects in the disclosed methods.

Samples can be exposed to one or more agents under a wide array of conditions. Some conditions which can, but not necessarily need to, influence observable responses of a sample to exposure to an agent include duration of exposure, incubation temperature, agent concentration, amount of sample, agent activation state, presence of additional factors (e.g. co-factors, substrates, enzymes, etc.), condition of samples (e.g. clumped vs. dispersed cells), and other variables. Preferably, the agent is exposed to samples for a time sufficient for a response to be observed and recorded.

Samples exposed to an agent can be assayed in numerous ways known to those of skill in the art. Assays can be designed to control for a particular condition, e.g. agent concentration. In some embodiments, the agent is exposed to replicates of samples in an array. For instance, various concentrations of the agent may be exposed to numerous sample replicates in a population study. More than one agent can be included in an array. As an example, samples can be exposed to a constant concentration of a first agent and increasing (or decreasing) concentrations of a second agent. Additional variables can be simultaneously tested in the same array. For instance, samples exposed to a constant concentration of a first agent and increasing (or decreasing) concentrations of a second agent can additionally be incubated at varying temperatures. Further, the methods can include samples from numerous populations and subpopulations. Thus, two or more subpopulations can be exposed to the agent (or agents) and assayed (under one or more conditions).

Methods disclosed herein can identify populations having different responses to agents. As used herein, a “response” or “reaction” refers to any observable change which occurs after exposing the agent to the population samples. The change can be in the type of response, magnitude of response, or combination thereof. Examples of responses which are changes in type include cells which normally produce one compound but produce another upon exposure to the agent, and living cells which die upon exposure to the agent. Examples of responses which are changes in magnitude include cells which produce a compound but produce less upon exposure to the agent, as well as percentage of cells which continue to live upon exposure to the agent. In some embodiments, sub-populations which are compared in the disclosed methods can comprise a substantially similar response, e.g. not different to a statistically significant degree. In some embodiments, sub-populations which are compared in the disclosed methods can comprise a statistically significant different response.

Responses observed in the methods should be evaluated, quantitatively or qualitatively, to develop meaningful conclusions based on the results of the methods. Quantitation of responses typically depends on the components involved and the nature of the exposure, as understood by one of skill in the art. For instance, agent toxicity to cell samples can be measured in various live/dead stains, metabolic assays, visualization and scoring (e.g. microscopy), and/or other experimental procedures. Endpoints of a response represent a means to compare the results of one sample/agent exposure to the results of another sample/agent exposure. Like selection of quantitation methods, selection of endpoints of a response typically depend on the nature of the study and the selected quantitation method. For instance, an endpoint of a response, as it relates to e.g. cellular toxicity, may be the percentage of metabolically active cells remaining after exposure to the agent for a period of one minute. As another example, an endpoint of a response, as it relates to e.g. cellular toxicity, may be the duration of exposure after which no observable change in metabolic activity occurs. While the former example can be evaluated using a single data point obtained at one minute post-exposure, the latter example can be evaluated using numerous data points over time, or alternatively, selecting a single data point from the numerous data points as representative of the observed response. Other means of quantitation such as averages or other derivative data are also anticipated.

Data derived from samples can be used in statistical analysis of population and subpopulation studies. For instance, endpoints obtained from samples exposed to an agent in an array can be analyzed as a distribution of endpoints. Distributions, inclusive of averages, means, trend lines, and other methods to compare bulk data, permit the identification of population trends, tendencies, and other characteristics. As such, the distribution of endpoints of a first sub-population of samples can be compared to the distribution of endpoints of a second sub-population of samples. Based on said comparison, a method user can determine whether the response of the first sub-population samples to exposure to the agent is statistically different from the response of the second sub-population samples to exposure to the agent.

The number of sub-populations used in a disclosed method is limited by employed statistical parameters and constraints such as the total available pool of samples from various subjects. Because subjects can include members of the same species, e.g. humans, from anywhere in the world, the pool of potential subjects is extensive. As the actual pool of subjects from which samples are collected increases in number, the array of selectable subpopulations increases in number and diversity. Thus, although the number of sub-populations used in a disclosed method is generally not limited, such numbers can be limited by the number and diversity of subjects in the overall populations from which samples are collected. As such, the methods can comprise at least 2 (e.g. a first and a second subpopulation), at least 3 (e.g. a first, a second, and a third subpopulation), at least 5, at least 10, or at least 20 subpopulations. The methods can comprise 2, 3, 4, 5, 10, 20, 50, or 100 subpopulations. Further, a subset of sub-populations in a study can be selected for comparison. For instance, numerous sub-populations can be exposed to an agent (or agents) and assayed, yet a method user may opt to compare a first and a second subpopulation; a first and a third subpopulation; a first, second, and third, subpopulation; and so on.

A user may choose to base conclusions as to the relationship, or differences, between the populations or sub-populations on any statistical, mathematical or graphical comparison of the results of the experiments. Statistical analysis of the data also depends on the nature of the study performed and is inclusive of a wide array of statistical studies, programs, and techniques known to those of skill in the art. Whether a difference is significant, or statistically significant, depends on the parameters selected for the method. In some embodiments, the threshold for statistical significance is determined by a statistical p-value. As non-limiting examples, any or all of the following could be used: comparisons of each sub-population's mean mode or medians; comparisons of any percentile of results (e.g. the 80th percentile) of the compared sub-populations; a notation that 44 percent of the observations for levels of effects on a first sub-population fall substantially below the lowest observed level of effect for any member of a second sub-population, etc. This list is illustrative, and is not intended to be comprehensive. Any applicable technique to evaluate the difference in responses of sub-populations to exposure to an agent may be used, as would be understood by one of skill in the art. Guidance for evaluating the significance of any differences in data can be found, for example, in Navidi, W. C. et al., Elementary Statistics, McGraw-Hill Higher Education (October 2014); and in Lane, D. et al., Online Statistics Education: A Multimedia Course of Study (available online http://onlinestatbook.com/) each of which are fully incorporated by reference herein.

Also disclosed are methods for determining whether a first subpopulation exposed to radiation with have a statistically significant difference in a response to exposure to a chemical or biological agent compared to a second subpopulation, comprising: a) exposing the chemical or biological agent to samples collected from subjects of a first sub-population exposed to toxic levels of radiation and samples collected from subjects of a second sub-population not exposed to toxic levels of radiation for a time sufficient for a response to be observed; b) assaying the samples of a) exposed to the agent for a response; c) quantifying an endpoint of the response for each sample; d) providing a distribution of endpoints quantified in step c) for each of the first and second sub-populations samples; e) comparing the distribution of endpoints of the first sub-population samples to the distribution of endpoints of the second sub-population samples; and f) determining whether the response of the first sub-population samples to exposure to the agent is statistically different from the response of the second sub-population samples to exposure to the agent.

The radiation can be from any source which produces levels of radiation toxic to a subject. The radiation can be from, for example, a leak from a container or containment facility, nuclear explosion and/or meltdown, so-called “dirty-bombs” which release radiation in the absence of nuclear fission or fusion, removal of natural and/or artificial barriers from existing radiation deposits, etc. In some embodiments, toxic levels of radiation are from a radiation release resulting in evacuation of at least some humans in local proximity to the radiation release. Well-known examples include the Fukushima and Chernobyl nuclear reactor disasters. Other examples include, but are not limited to, radiation spills or leaks, whether accidental or intentional, in laboratories, factories, and other institutions which harbor or use radioactive materials. In some embodiments, the radiation can be from multiple sources.

The radiation, in some embodiments, can be ionizing radiation including, but not limited to, x-ray radiation, y-radiation, energetic electron radiation, ultraviolet radiation, thermal radiation, cosmic radiation, electromagnetic radiation, nuclear radiation, or any combination thereof.

The exposure to toxic levels of radiation can be acute, intermittent or prolonged. Both the amount and the rate of exposure affect the level of toxicity of the exposure. For instance, prolonged or intermittent exposures to low levels of radiation over time can result in toxicity in the same manner that acute exposure to high levels of radiation. A single dose of radiation delivered in one acute exposure can result in greater toxicity than the exposure to the same amount over large periods of time. In some embodiments, the exposure can be in the form of ingestion, inhalation, absorption, adsorption, injection, etc., and any combination thereof.

Toxic levels of radiation include any amounts of radiation which, when exposed to a subject either acutely, intermittently, or in small doses over prolonged periods of time, can cause toxic effects in the subject. Typically, humans can tolerate up to about 5 rem annually without significant toxicity. However, exposures of 5-20 rem can cause temporary blood changes fetal microencephaly, oligospermia, and increase the risk for cancer and genetic effects. See Health and Hazardous Waste, New Jersey Dept. of Health, 1(3): 1996. 350 to 500 rem can result in lethal doses to 50 percent of a human population within 30 days. Id. In some embodiments, the toxic level of radiation is at least 1 rem, 5 rem, at least 10 rem, at least 15 rem, at least 20 rem, at least 25 rem, at least 30 rem, at least 40 rem, at least 50 rem, at least 60 rem, at least 70 rem, at least 80 rem, at least 90 rem, or at least 100 rem.

Also disclosed herein are methods for determining whether race or ethnicity of a first subpopulation with result in a statistically significant difference in a response to exposure to a chemical or biological agent compared to a second subpopulation, comprising: a) exposing the chemical or biological agent to samples collected from subjects of a first sub-population and samples collected from subjects of a second sub-population for a time sufficient for a response to be observed, wherein the subjects of the first sub-population differ in race or ethnicity from the subjects of the second sub-population; b) assaying the samples of a) exposed to the agent for a response; c) quantifying an endpoint of the response for each sample; d) providing a distribution of endpoints quantified in step c) for each of the first and second sub-populations samples; e) comparing the distribution of endpoints of the first sub-population samples to the distribution of endpoints of the second sub-population samples; and f) determining whether the response of the first sub-population samples to exposure to the agent is statistically different from the response of the second sub-population samples to exposure to the agent.

In some embodiments, race and/or ethnicity can be related to country of residency. As such, the residents of one country can comprise subjects of a particular set of races and/or ethnicities, whereas the residents of another country (or multiple other countries) can comprise subjects of a different set of races and/or ethnicities. In some embodiments, residents of two or more countries can comprise subjects of the same, or substantially similar, races and/or ethnicities. In some embodiments, the first subpopulation comprises residents of one or more countries that have not received regulatory approval for the agent. In some embodiments, the second subpopulation comprises residents of one or more countries that have received regulatory approval for the agent.

Also disclosed herein are methods for identifying whether two or more populations have a statistically significant difference from each other in a response to a chemical or biological agent, comprising a) providing sample cells that can be reprogrammed into iPSCs from a sample of subjects of a first sub-population who possess one or more phenotypic or genotypic traits suspected of causing or being co-incidentally related to one or more biological reactions to the chemical or biological agent; b) providing sample cells that can be reprogrammed into iPSCs from a sample of subjects of a second sub-population having one or more phenotypic or genotypic traits that are different from those of the first sub-population, and wherein these traits are suspected of causing or being co-incidentally related to one or more biological reactions to the chemical or biological agent; c) optionally providing sample cells that can be reprogrammed into iPSCs from a sample of subjects of additional sub-populations that meet the same criteria as in step b); d) reprogramming the cells from each subject of the sub-populations in a) through c) into iPSCs, and optionally, further differentiating the iPSCs into functional cells; e) for each sample from each subject, combining the chemical or biological agent in vitro with the cells developed in step d); f) assaying for an effect of result of the reaction for each member of each sub-population; g) quantifying an endpoint of the effect of each sample; h) comparing the distribution of endpoint scores of the effect on subjects from the first sub-population to the distribution of endpoints of the effect on subjects from the second sub-population, and, optionally, any additional sub-populations; and i) employing statistical techniques to determine whether the effect of the chemical or biological agent on subject samples from the first sub-population are statistically different from the effect on subject samples from the second and/or additional sub-populations.

Disclosed herein are methods which can be used with an array of materials useful for carrying out any one or more disclosed method. Where a method is disclosed and a number of modifications to the method are discussed, each and every combination and permutation of the method, and the modifications that are possible, are specifically contemplated unless specifically indicated to the contrary. Likewise, any subset or combination of these is also specifically contemplated and disclosed. This concept applies to all aspects of this disclosure. Thus, if there are a variety of additional steps that can be performed, it is understood that each additional step can be performed with any specific method step or combination of method steps of the disclosed methods, and in any order or permutation, unless otherwise indicated, and that each such combination or subset of combinations is specifically contemplated.

Publications cited herein are hereby specifically incorporated by reference in their entireties and at least for the material for which they are cited.

### EXAMPLES

The examples below are intended to further illustrate certain aspects of the methods and compositions described herein, and are not intended to limit the scope of the claims.

Example 1: Using a pre-existing sample of donor tissues to search for novel distinctions in the reactions of sub-populations. A researcher creates a large scale (e.g., 100 person) representative sample of a population, wherein the sample consists of iPSCs derived from a single cell type. The process of selecting appropriate donors is well known to those skilled in the art. Processes involved in selecting the most appropriate donor cells, collecting them, converting them into iPSCs, and culturing and differentiating these cells can be found in PCT/US14/45499, entitled “Methods for Predicting Responses to Chemical or Biologic Substances”; PCT/US14/53819, entitled “Methods for Genetically Diversified Stimulus-Response Based Gene Association Studies”; and PCT/US15/55637, entitled “Methods for Conducting Stimulus-Response Studies with Induced Pluripotent Stem Cells Derived from Perinatal Cells or Tissues”. The researcher then genetically sequences either portions of the genome or the whole genome from each of the samples and stores the results for later use.

The researcher then conducts a set of assays (each involving multiple endpoints) on all the members of the sample to test for the effects of a compound or set of compounds. The data is analyzed and stored.

At later times, the researcher uses the above platform to assist other scientists in attempts to learn whether certain genotypes may partially or fully explain differences in reactions among humans, through two related analytical and experimental techniques.

Subsequently, a requesting scientist identifies differing alleles of a particular gene—one having relatively few alleles (with the rarer or rarest allele to participate in the analysis having a prevalence in the sample of at least 10 donors)—to be associated with different levels of reaction to a chemical agent of interest (as measured by a particular endpoint of a particular assay that the researcher conducted above). Here, the researcher uses the genetic information obtained from the sequencing analysis above to subdivide the sample by the gene alleles of interest. The researcher then assembles the relevant endpoint data from the earlier assay conducted on the agent of interest, and divides the data based on the membership of each subdivision. A distribution of results (showing severity or intensity of biological reaction) is created for each subdivision, and the distributions compared using ANOVA and other quantitative techniques for comparing distributions. In this way, the researcher is able to ascertain whether the genetically-defined subdivisions have statistically relevant differing reactions.

In the second technique, the requesting scientist defines the subdivisions of the sample via any genotypic or phenotypic criterion for which information is available to assign each individual to the correct sub-group, but the requesting scientist seeks reaction information that has not been previously gathered by the researcher. In this embodiment, the researcher must conduct additional assays to develop the endpoint data for each individual anew. Thereafter, the technique is the same as for the first technique.

Example 2: Testing for the impact of exposure to radiation. Following the leakage of radiation from the Fukushima Power Plant in Japan, a group of researchers conducts studies to ascertain whether victims' exposure to radiation will likely affect their reactions to pharmaceuticals in the future, such that doctors prescribing such pharmaceuticals might need to either change the prescription protocols when treating these individuals, or change patient monitoring practices when treating these individuals.

Given the need to test a variety of compound classes, and the ethical issues involved, in vivo studies are not contemplated. Further, given the previous suffering of the victims and the invasiveness of various tissue collection procedures, the researchers choose to take only a single tissue sample from each victim selected for the study. These considerations lead the researchers to choose iPSCs derived from dermal fibroblasts (and cells derived from those iPSCs) as the cell platform on which the relevant assays are to be conducted. Therefore, a single skin samples is collected from each victim participating in the study.

After the case samples representing those who have been affected by the radiation leak have been defined, individuals matching the selection criteria are identified and tissue samples are taken from those individuals according to protocols well known by those skilled in the art. The tissue samples are converted into iPSCs, using a commercial kit such as that available from Reprocell-Stemgent (Lexington, Mass.). These iPSCs and their derivative cells are designated as the case sample.

A control sample containing iPSCs (and their derivatives) taken from members of the Japanese society who were not exposed to the radiation, but who broadly match the case sample in other respects (e.g., age, gender, medical histories prior to the exposure) are collected.

All members of both the case and control sample are exposed (in predefined assays) to identical dose concentrations of a variety of compounds that the researchers suspect may behave differently in the case population than in the control population.

The endpoints from the assays are arithmetically manipulated to form two data sets: Data Set One consisting of the absolute endpoint scores at the dose concentration of interest, and Data Set Two measuring—for each individual—the difference between the end point score when challenged by a compound at the dose concentration of interest and the endpoint score when that same individual is challenged only by a control compound known to have zero effect on the test samples.

By comparing the distributions of endpoints from either data set using various statistical methods (such as ANOVA), the researchers are able to determine which compounds produce different effects in individuals (as measured by endpoints that relate to severity or intensity of biological reaction) who have been exposed to the radiation leak versus those who have not been exposed. Thus, further research can be focused on compounds showing differential impact between the sample of persons exposed to the radiation and the control sample.

Example 3: Testing for the impact of ethnicity. The Ministry of Health in a medium-sized Southeast Asian country (other than Japan) is concerned that its citizens may experience cardiotoxic reactions to certain pharmaceuticals, even though those same pharmaceuticals have been approved by the U.S. FDA and the Pharmaceuticals and Medical Devices Agency in Japan. At issue is whether the racial/ethnic differences between the country's citizens and those of the U.S. and Japan might result in different toxic reactions. (Race has been previously identified in numerous academic papers as a significant contributor to differences in toxicity reactions.)

Unfortunately, the country is not in a position to demand that the pharmaceutical companies conduct clinical trials on a sample of that country's citizens because the size of the local market is too small to justify the significant expense of carrying out such trials.

Therefore, the agency chooses to test for such effects in vitro. However, the agency recognizes that the state of the art of in vitro toxicology testing (including cardiotoxicity) is not yet developed to the point that researchers can directly translate a particular numerical score for an endpoint when measured in vitro to a quantitative prediction of the severity of impact in vivo. (For example, there are no validated translations that would take the form, “If the score for an individual on this endpoint in this assay is 87, then the probability of this individual experiencing arrhythmia under analogous circumstances in vivo is approximately 6 percent.”)

Instead, the agency chooses to compare the impact of the pharmaceuticals of interest on iPSCs and derivative cells created from cells taken from a large sample of its citizens to the impact of those same pharmaceuticals on large samples of residents of other countries that have received regulatory approval of the compounds after conducting clinical trials (and have subsequently experienced only acceptably low incidence and severity of cardiotoxic side effects). It decides that the “populations” to be compared are (1) its indigenous population, (2) the U.S. population, and (3) the Japanese population.

A service provider has previously developed large scale samples of the U.S. and Japanese populations, using Endothelial Progenitor Cells taken from cord blood. Under contract, the service provider creates an analogous sample for the client country's indigenous population. The same provider then administers a compound or compounds of interest to the three populations, assays for cardiotoxicity in each sample, and records the scores for the various endpoints for each individual tested.

The scores for each endpoint are then aggregated across all of the members of a particular sample, forming a distribution of the “severity” experienced by that sample. Once such distributions have been developed for all three samples, a variety of statistical tests, including an ANOVA comparison, are conducted to determine whether the indigenous population's responses (as represented by its sample) are different (i.e., better or worse, and to what degree) than the U.S. or Japanese populations (again as represented by their samples).

For most compounds, the Ministry finds that any differences among the population results are reassuring (that is, that the indigenous population scores no worse, or statistically insignificantly worse) than at least one of the two comparison countries. These compounds are, therefore, given regulatory approval in the client's country.

In the cases of those compounds for which some endpoint results were significantly worse in the case of the indigenous sample than either the U.S. or Japanese samples, the regulators open negotiations on the extent and nature of steps (such as targeted clinical safety trials, warning labels, etc.) that the drug companies must undertake before seeking approval.

1. A method for identifying two or more populations having a difference in a response to exposure to a chemical or biological agent, comprising:
a) exposing the chemical or biological agent to samples collected from subjects of a first sub-population and samples collected from subjects of a second sub-population for a time sufficient for a response to be observed,
wherein one or more phenotypic or genotypic traits of the subjects of the first sub-population are different from the phenotypic and genotypic traits of the second sub-population and are suspected of being related to one or more responses to exposure to the agent;
b) assaying the samples of a) exposed to the agent for a response;
c) quantifying an endpoint of the response for each sample;
d) providing a distribution of endpoints quantified in step c) for each of the first and second sub-populations samples;
e) comparing the distribution of endpoints of the first sub-population samples to the distribution of endpoints of the second sub-population samples; and
f) determining whether the response of the first sub-population samples to exposure to the agent is different from the response of the second sub-population samples to exposure to the agent.

2. The method of claim 1, wherein the first and second sub-populations are sufficiently similar such that a difference in the response to exposure to the agent is primarily due to the one or more phenotypic or genotypic traits of the subjects of the first sub-population that are different from the phenotypic and genotypic traits of the second sub-population and are suspected of being related to one or more responses to exposure to the agent.

3. The method of claim 1, wherein the samples collected from subjects of the first and second sub-populations comprise cells that are reprogrammed in vitro to induced pluripotent stem cells.

4. The method of claim 3, further comprising differentiating the induced pluripotent stem cells into functional cells.

5. The method of claim 1, wherein the subjects are humans.

6. The method of claim 1, wherein more than about 10 samples collected from subjects of a first sub-population and more than about 10 samples collected from subjects of a second sub-population are exposed to the agent.

7. The method of claim 1, wherein more than one agent is exposed to the samples and assayed.

8. The method of claim 1, wherein any of two or more subpopulations are exposed to the agent and assayed.

9. The method of claim 1, wherein each subject is classified as being within the first or the second sub-population based on differing alleles of at least one gene.

10-17. (canceled)

18. A method for determining whether race or ethnicity of a first subpopulation with result in a difference in a response to exposure to a chemical or biological agent compared to a second subpopulation, comprising:
a) exposing the chemical or biological agent to samples collected from subjects of a first sub-population and samples collected from subjects of a second sub-population for a time sufficient for a response to be observed,
wherein the subjects of the first sub-population differ in race or ethnicity from the subjects of the second sub-population;
a) assaying the samples of a) exposed to the agent for a response;
b) quantifying an endpoint of the response for each sample;
c) providing a distribution of endpoints quantified in step c) for each of the first and second sub-populations samples;
d) comparing the distribution of endpoints of the first sub-population samples to the distribution of endpoints of the second sub-population samples; and
e) determining whether the response of the first sub-population samples to exposure to the agent is different from the response of the second sub-population samples to exposure to the agent.

19. The method of claim 18, wherein the first subpopulation comprises residents of one or more countries that have not received regulatory approval for the agent.

20. The method of claim 18, wherein the second subpopulation comprises residents of one or more countries that have received regulatory approval for the agent.

21. The method of claim 18, wherein the response comprises a cardiotoxicity response.

22. The method of claim 18, wherein the samples collected from subjects of the first and second sub-populations comprise cells that are reprogrammed in vitro to induced pluripotent stem cells.

23. The method of claim 22, further comprising differentiating the induced pluripotent stem cells into functional cells.

24. The method of claim 18, wherein the subjects are humans.

25. The method of claim 18, wherein more than about 10 samples collected from subjects of a first sub-population and more than about 10 samples collected from subjects of a second sub-population are exposed to the agent.

26. The method of claim 18, wherein more than one agent is exposed to the samples and assayed.

27. The method of claim 18, wherein any of two or more subpopulations are exposed to the agent and assayed.

28. A method for identifying whether two or more populations have a statistically significant difference from each other in a response to a chemical or biological agent, comprising:
a) providing sample cells that can be reprogrammed into iPSCs from a sample of subjects of a first sub-population who possess one or more phenotypic or genotypic traits suspected of causing or being co-incidentally related to one or more biological reactions to the chemical or biological agent;
b) providing sample cells that can be reprogrammed into iPSCs from a sample of subjects of a second sub-population having one or more phenotypic or genotypic traits that are different from those of the first sub-population, and wherein these traits are suspected of causing or being co-incidentally related to one or more biological reactions to the chemical or biological agent;
c) optionally providing sample cells that can be reprogrammed into iPSCs from a sample of subjects of additional sub-populations that meet the same criteria as in step b);
d) reprogramming the cells from each subject of the sub-populations in a) through c) into iPSCs, and optionally, further differentiating the iPSCs into functional cells;
e) for each sample from each subject, combining the chemical or biological agent in vitro with the cells developed in step d);
f) assaying for an effect of result of the reaction for each member of each sub-population;
g) quantifying an endpoint of the effect of each sample;
h) comparing the distribution of endpoint scores of the effect on subjects from the first sub-population to the distribution of endpoints of the effect on subjects from the second sub-population, and, optionally, any additional sub-populations; and
i) employing statistical techniques to determine whether the effect of the chemical or biological agent on subject samples from the first sub-population are statistically different from the effect on subject samples from the second and/or additional sub-populations.
